(19) United States
(12) Patent Application Publication
(10) Pub. No.: US 2005/0059132 A1
(43) Pub. Date: Mar. 17, 2005

(54) Protein C variants

(76) Inventors: Bjorn Dahlback, Malmo (SE); Gary L. Nelsestuen, St. Paul, MN (US)

Correspondence Address: BIRCH STEWART KOLASCH & BIRCH, PO BOX 747, FALLS CHURCH, VA, 22040-0747, US

(21) Appl. No.: 10/652,947

(22) Filed: Sep. 2, 2003

Related U.S. Application Data
(63) Continuation of application No. PCT/SE02/00363, filed on Mar. 1, 2002.
(60) Provisional application No. 60/272,466, filed on Mar. 2, 2001.

(51) Int. Cl. C12N 9/74; C07H 21/04; C07K 14/745

(52) U.S. Cl. 435/214; 435/69.1; 435/320.1; 435/325; 536/23.2; 530/383

(57) ABSTRACT

The present invention is concerned with a variant blood coagulation component, which is substantially homologous in amino acid sequences to a wild-type blood coagulation component capable of exhibiting anticoagulant activity in the protein C-anticoagulant system of blood and selected from protein C (PC) and activated protein C (APC), said variant component being capable of exhibiting an anticoagulant activity, that is enhanced in comparison with anticoagulant activity expressed by the corresponding wild-type blood coagulation component, said variant component differing from the respective wild-type component, in that it contains in comparison with said wild-type component at least one amino acid residue modification in it N-terminal amino acid residue sequence that constitutes the Gla-domain of protein C. The present invention is also concerned with methods to produce such variants based on DNA technology, with DNA segments intended for use in the said methods, and with use of said variants for therapeutic and diagnostic purposes.

### CROSS REFERENCE TO RELATED APPLICATIONS

This application is a continuation of PCT International Application No. PCT/SE02/00363 which has an International filing date of Mar. 1, 2002, which designated the United States of America. This application claims priority under 35 U.S.C. §119 on provisional application No. 60/272,466 filed in the United States of America on Mar. 2, 2001. Both of the above applications are incorporated by reference in their entirety.

### FIELD OF THE INVENTION

The present invention is directed to functional recombinant protein C variants expressing enhanced anticoagulant activity, and to use of such variants for therapeutic or diagnostic purposes. More specifically, the present invention is directed to protein C variants containing a modified Gla-domain and to use of such variants for therapeutic or diagnostic purposes.

### BACKGROUND OF THE INVENTION

Protein C is a vitamin K-dependent protein of major physiological importance that participates in an anticoagulant system of the blood, which is generally designated the protein C-anticoagulant system. Like all vitamin K-dependent proteins, protein C contains a Gla-domain or Gla-module that is comprised of the N-terminal 45 amino acid residues, said domain being crucial for membrane binding-affinity as will be discussed in more detail below.

In said protein C-anticoagulant system, protein C functions in concert with other proteins including the cofactors protein S and intact Factor V (FV), which act as synergistic cofactors to protein C in its activated form (APC, Activated Protein C), as a down-regulator of blood coagulation hereby preventing excess coagulation of blood and, thus, inhibiting thrombosis. This anticoagulant activity that is expressed by the activated form of protein C emanates from its capacity to inhibit the reactions of blood coagulation by specifically cleaving and degrading activated Factor VIII (FVIIIa) and activated Factor V (FVa), these being other cofactors of the blood coagulation system. As a result thereof, activation of components necessary for blood coagulation, viz. Factor X (FX) and prothrombin, is inhibited and the activity of the coagulation system is dampered. Protein C is, thus, of major physiological importance for a properly functioning blood coagulation system.

The importance of protein C can be deduced from clinical observations. For instance, severe thromboembolism affects individuals with homozygous protein C deficiency and affected individuals develop thrombosis already in their neonatal life. The resulting clinical condition, purpura fulminans, is usually fatal unless the condition is treated with protein C. On the other hand, heterozygous protein C deficiency is associated with a less severe thromboembolic phenotype and constitutes only a relatively mild risk factor for venous thrombosis. It has been estimated that carriers of this genetic trait have a 5- to 10-fold higher risk of thrombosis as compared to individuals with normal protein C levels.3:35 PM 2/16/2005 More importantly, however, the most common genetic defect associated with thrombosis is also affecting the protein C system. This condition is usually referred to as APC resistance and is most frequently caused by a single point mutation in the FV-gene, which mutation leads to replacement of the amino acid residue Arg506 with a Gln residue in the FV amino acid sequence. Arg506 constitutes one of three cleavage sites in activated FV (FVa), which are sensitive to cleavage action by APC, and such mutated FVa is less efficiently degraded by APC than normal FVa (Dahlbäck, J. Clin. Invest. 1994, 94: 923-927).

The physiological importance of protein C and activated protein C (APC) as anticoagulant components in the blood coagulation system indicate potential use of these substances for therapeutic purposes.

Indeed, protein C and its activated form APC have already been used to some extent for therapeutic purposes (Verstraete and Zoldholyi, Drugs 1995, 49: 856-884; Esmon et al, Dev. Biol. Stand. 1987, 67: 51-57; Okajima et al, Am. J. Hematol. 1990,33: 277-278; Dreyfys et al, N. Engl. J. Med. 1991, 325: 1565-1568). More specifically, protein C purified from human plasma has been used as replacement therapy in homozygous protein C deficiency (Marlar and Neumann, Semin. Thromb. Haemostas. 1990, 16: 299-309) and has also been used successfully in cases with severe disseminated intravascular coagulation due to meningococcemia (Rivard et al, J. Pediatr. 1995, 126: 646-652). Moreover, in a baboon model of septicaemia (using E. coli), APC was shown to have a protective effect, which was particularly pronounced when the APC was given prior to the E. coli infusion (Taylor et al, J. Clin. Invest. 1987, 79: 918-925). In any event, the results obtained to date suggest that protein C may become a useful drug, not only for treatment of the above conditions but also for many other conditions, in which the coagulation system is activated, e.g. for the prevention and treatment of venous thrombosis, vascular occlusion after recanalization of coronary vessel after myocardial infarction (MI) and after angioplasty.

It is envisioned that therapeutic treatment of various conditions related to blood coagulation disturbances could be improved if variants of protein C having enhanced anticoagulant properties were available. Moreover, such variants would be useful as reagents to improve various biological assays for other components of the protein C system in order to obtain assays having improved performance.

The development of recombinant DNA technology in the past decades has had a tremendous impact on the possibilities to produce desired biological substances efficiently and/or to create biological substances having desired and optionally specifically designed properties. Indeed, not only functional variants of protein C but also essentially wild-type protein C have been produced by recombinant technology, e.g. as reported in the following references.

In U.S. Pat. No. 4,775,624 (Bang et al) recombinant production of human protein C derivatives is disclosed. However, only production of protein C polypeptides having functional activities essentially corresponding to human wild-type protein C is disclosed. Recently, wild-type protein C produced in accordance with this reference has been used successfully in treatment of severe sepsis (cf. a release before publication of an article with the title “Efficacy and Safety of Recombinant Human Activated Protein C for Severe Sepsis” from New England Journal of Medicine and dated Feb. 9, 2001).

Use of protein C prepared by recombinant technique has also been disclosed in Berg et al, Biotechnique, 1993, 14: 972-978; Hoyer et al, Vox Sang. 1994, 67: Suppl. 3: 217-220).

Moreover, functional variants of protein C obtained by mutagenesis directed to the activation peptide region, which includes residues 158-169, may have enhanced sensitivity to thrombin, such variants being activated by thrombin faster than wild-type protein C (Erlich et al, Embo. J. 1990, 9: 2367-2373; Richardson et al., Nature 1992, 360:261-264). In one of these studies (Richardson et al., Nature 1992, 360: 261-264), a number of mutations were introduced around the activation site leading to a mutant protein C, that was relatively easily activated by thrombin formed during coagulation of blood even in absence of thrombo-modulin, a membrane protein, that is usually required for efficient activation of protein C by thrombin.

More specifically, those protein C variants having enhanced interaction with thrombin that are disclosed in Richardson et al., Nature, 1992, 360:261-264, comprise mutations in the activation peptide region, two putative inhibitory acidic residues near the thrombin cleavage site being altered. One protein C variant comprising said altered residues in the activation peptide region and also the Asn313Gln mutation disclosed by Grinnell et al. (infra) has recently been shown to function well as an anticoagulant in experiments performed in vivo (Kurz et al., Blood, 1997, 89: 534-540). However, in this protein C variant the enhanced anticoagulant activity is due to the Asn 313 Gln mutation, the other mutations giving rise to enhanced interaction with thrombin.

In Grinnell et al., J. Biol. Chem., 1991, 9778-9785, the role of glycosylation in the function of human protein C is examined, site-directed mutagenesis being used to singly eliminate each of the four potential N-linked glycosylation sites, i.e. the positions 97, 248, 313 and 329. In the protein C variants disclosed therein, Gln is substituted for Asn at positions 97, 248 and 313, resp., and it is shown, that the protein C mutants having this substitution mutation at positions 248 and 313 expressed a 2- to 3-fold enhanced anticoagulant activity in addition to other modified properties.

Functional variants of protein C and APC that exhibit enhanced anticoagulant activity due to introduction of at least one amino acid residue modification in the amino acid sequence of wild-type protein C, e.g. in the serine protease (SP) module, which modification does not alter the glycosylation of protein C, are disclosed in WO 98/44000. One variant specifically disclosed therein contains a few mutations in the SP module that are located within a short amino acid residue stretch between the residue nos. 300 and 314, said variant exhibiting approximately 400% enhanced anticoagulant activity as compared to wild-type human protein C.

In J. Biol. Chem. 1993, 268; 19943-19948 Rezaie et al. disclose a protein C mutant comprising a Glu357Gln mutation (i.e. Glu192Gln if chymotrypsin numbering is used). Although this mutant inactivates FVa at an about 2- to 3-fold enhanced rate in a pure system, in plasma the anticoagulant activity is not enhanced as compared to wild-type protein C since the mutant is rapidly inhibited by protease inhibitors such as alpha-1 antitrypsin and antithrombin III.

Protein C variants having modifications in or lacking the Gla-domain of native protein C have also been reported previously.

For instance, a protein C variant lacking the Gla-domain of native protein C and comprising a Thr254Tyr (i.e. Thr99Tyr based on the chymotrypsin numbering) is disclosed in J. Biol. Chem., 1996, 271: 23807-23814. This variant protein C has a 2-fold enhanced activity towards pure FVa, i.e. soluble FVa in absence of phospholipids, but is lacking anticoagulant activity in plasma by virtue of the missing Gla-domain.

Recently, a few protein C variants having a modified Gla-domain have been reported by Shen et al. in J. Biol. Chem., Vol. 273, No. 47, pp. 31086-31091, 1998. These protein C variants contain a few substitutions in the Gla-domain and exhibit enhanced Ca and/or membrane binding properties and, thus, also enhanced anticoagulant activity of activated protein C (APC). Some of these variants have also been disclosed in WO 99/20767 together with other protein C variants containing substitution modifications in the Gla-domain. The latter reference is generally related to modified vitamin K-dependent polypeptides exhibiting altered, e.g. enhanced, membrane binding-affinity due to modifications, i.e. substitutions, in their Gla-domains. The vitamin K-dependent polypeptide could comprise factor VII or any other vitamin K-dependent protein, e.g. protein C. It is to be noted that the numbering of the Gla-domain residues differs between Shen et al. and this WO reference in that according to the WO reference position 4 in the protein C sequence is not occupied by any residue, which means that e.g. position 10 according to Shen (and the present invention) corresponds to position 11 according to the WO reference.

Even though protein C variants having enhanced anticoagulant activity and/or other modified properties have been disclosed previously, there is still a need of protein C variants that exhibit enhanced anticoagulant activity and/or have other beneficial properties that would be useful for therapeutic and/or diagnostic purposes.

### SUMMARY OF THE INVENTION

The present invention is concerned with functional variants of protein C, that contain a modified Gla-domain and exhibit enhanced anticoagulant activity. This enhanced anti-coagulant activity of the present protein C its emanates essentially from enhanced calcium and/or membrane binding properties and is mainly expressed by APC, which is the active form of the protein C zymogen, said zymogen being virtually inactive. Accordingly, the present invention is also concerned with variants of APC that contain a modified Gla-domain and exhibit enhanced anticoagulant activity. The Gla-domain comprises the first amino-terminal 45 residues of protein C and its structure and function will be discussed in more detail below.

According to the present invention it has been discovered that introduction of at least one, suitably a few, e.g. at least 6, and more specifically 7 or more, amino acid residue modification(s) into the Gla-domain, provides protein C or APC variants that have improved properties as compared to the variants having modifications in the Gla-domain that have been reported by Shen et al. (loc. cit.) and in the WO 99/20767 publication.

Suitably, the present variants do not contain more than 10 amino acid modifications and, suitably, do not encompass hybrids between different vitamin K-dependent proteins, such as hybrid protein C variants having a Gla-domain derived from prothrombin or Factor X, unless the differences between this other Gla-domain and the Gla-domain of protein C only constitute a few amino acid residues as discussed above.

Protein C variants according to the present invention that display improved properties, such as further enhanced anticoagulant activity, could provide benefits, e.g. by lowering the dosage or frequency of administration when used for therapeutic purposes.

The present invention is also concerned with methods to produce such variants based on DNA technology, with DNA segments intended for use in said methods, and with use of said variants for therapeutic and/or diagnostic purposes.

### BRIEF DESCRIPTION OF THE DRAWINGS

In the following, the present invention is disclosed in more detail with reference to the drawings, wherein:

FIG. 1 illustrates the effect of various APC variants (mutants) on the activated partial thromboplastin times (APTT) in human plasma. The following APC variants were examined: human wild-type (wt) APC (•), APC mutant QGN (□), APC mutant QGED (▴), APC mutant GNED (x), APC mutant SEDY (|) and APC mutant ALL (or QGNSEDY) (Δ).

FIG. 2 illustrates impact of human protein S on the effect of APC (wt and mutant) in an APTT assay. The following APC variants were examined: wt APC (•) and APC mutant QGNSEDY (ALL) (□).

FIG. 3 illustrates the effect of various APC variants on the prothrombin times in human plasma. The following APC variants were examined: wt APC (•), APC mutant QGN (□), APC mutant QGED (▴), APC mutant GNED (x), APC mutant SEDY (|), and APC mutant QGNSEDY (ALL) (Δ).

FIG. 4 illustrates the capacity of various APC variants to inactivate human factor Va as measured by the thrombin generation due to FXa-mediated activation of prothrombin, said activation being potentiated by FVa. The following APC variants were examined: wt APC (•), APC mutant QGN (□), APC mutant SEDY (+), and APC mutant QGNSEDY (ALL) (Δ).

FIG. 5 illustrates the capacity of various APC variants to inactivate human factor Va, the activity of FVa being measured with a prothrombinase assay. The following APC variants were examined: wt APC (•), APC mutant QGN (▴), APC mutant SEDY (Δ), and APC mutant QGNSEDY (ALL) (□) .

FIG. 6 illustrates inactivation of normal, i.e. wild-type (wt), FVa and Q506 mutant FVa (FVa Leiden) by APC. Values are shown for inactivation of: wt Eva with wt APC (•); wt FVa with APC mutant QGNSEDY (ALL) (□); R506Q FVa with wt APC (▴); and R506Q FVa with APC mutant QGNSEDY (ALL) (x).

FIG. 7-9 illustrate the ability of wt and variant protein C to bind to phospho-membranes. A surface plasma resonance technique from BIAcore was used. In these figures, different phospholipids were used, viz. 100% phosphatidylcholine (FIG. 7); a mixture of 20% phosphatidylserine and 80% phosphatidylcholine (FIG. 8); and a mixture of 20% phosphatidylserine, 20% phosphatidylethanolamine and 60% phosphatidylcholine (FIG. 9). In all tests, human wild-type protein C (wt) and the APC variants QGNSEDY (ALL), SEDY, SED, and QGN, were analyzed.

### DETAILED DESCRIPTION OF THE INVENTION

A. Molecular Arrangement of Protein C

The protein C molecule is composed of four different types of modules. In the direction of amino terminus to carboxy terminus, these modules consist of a Gla-module, two EGF-like modules, i.e. Epidermal Growth Factor homologous modules, and finally a typical serine protease (SP) module. In plasma, most of the circulating protein C consists of the mature two-chain, disulfide-linked protein C zymogen arisen from a single chain precursor by limited proteolysis. These two chains are the 20 kDa light chain, which contains the Gla- and EGF-modules and the 40 kDa heavy chain, which constitutes the SP-module. During activation by thrombin bound to thrombomodulin, a peptide bond Arg-Leu (residues 169 and 170) is cleaved in the N-terminal part of the heavy chain and an activation peptide comprising twelve amino acid residues (residues 158-169) is released. In connection with the present invention, the numbering of residues in the amino acid sequence of protein C and variants thereof is based on mature protein C.

The amino acid sequence of protein C has been deduced from the corresponding cDNA-nucleotide sequence and has been reported in the literature. Moreover, the cDNA-nucleotide sequences and the corresponding amino acid sequences for protein C are available from the EMBL Gene database (Heidelberg, Germany) under the accession number X02750 for human protein C, which is designated HSPROTC, and the accession number KO 2435 for bovine protein C, which is designated BTPBC.

As stated above, the Gla-domain of the vitamin K-dependent proteins comprises the N-terminal 45 amino acid residues. Thus, the amino acid sequence of the entire Gla-domain is known for proteins, such as human and bovine protein C, for which the entire amino acid sequence or the N-terminal part thereof (45 residues) has been determined. Based on the above database sequences, the Gla-domain of human protein C and bovine protein C can be illustrated as shown below (SEQ ID NO: 1 and SEQ ID NO:2, respectively);

| | |
|---|---|
| ANSFLEELRH SSLERECIEE ICDFEEAKEI | (SEQ ID NO:1) |
| FQNVDDTLAF WSKHV | |
| ANSFLEELRP GNVERECSEE VCEFEEAREI | (SEQ ID NO:2) |
| FQNTEDTMAF WSKYS | |

A comparison of such N-terminal sequences as regards similarities as well as deviations between individual sequences could indicate positions suitable as mutagenesis (i.e. modification) targets. For such a comparison it may not be necessary to know the entire amino acid sequence of the Gla-domain but it could be sufficient if the amino acid residues at positions potentially important for anticoagulant activity have been determined.

In connection with the present invention, the usual 1-letter or 3-letter symbols are used as abbreviations for amino acids as is shown in the following table of correspondence:

TABLE OF CORRESPONDENCE
SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| Z | Glx | Glu and/or Gln |
| W | Trp | tryptophan |
| R | Arg | arginine |
| D | Asp | aspartic acid |
| N | Asn | asparagine |
| B | Asx | Asn and/or Asp |
| C | Cys | cysteine |
| J | Xaa | Unknown or other |

B. Variants of Protein C

As stated above, the present invention is concerned with functional variants of recombinant protein C, said variants containing a modified Gla-domain and displaying enhanced anticoagulant activity. These variants differ from wild-type recombinant protein C as regards one or more, suitably a few and preferably not more than ten, amino acid residues, said residues being inserted, deleted or substituted (i.e. replaced) in the corresponding wild-type sequence, thereby giving rise to the present variants of protein C. Since said difference(s) is (are) maintained after activation of protein C to APC, the present invention is also concerned with APC variants having enhanced anticoagulant activity. According to a suitable embodiment the modification(s) is (are) substitution(s).

At present, such variants are conveniently obtained by mutagenesis, especially site-directed mutagenesis including use of oligonucleotide primers. However, the present invention is concerned with the functional variants per se irrespective of the mode of obtaining these variants.

In view of the close relationship between PC and APC, frequently, no clear distinction is made between PC and APC in connection with the present invention, but the designation PC/APC is used and the context will reveal if one or both of these substances are considered.

In connection with the present invention, the expression “variant” means a modified wild-type molecule, such as a mutant molecule, that generally has a high degree of homology as compared to the wild-type molecule.

Thus, it is preferred that such variants encompass only a few modified amino acid residues, and possibly only one amino acid residue, in order to preserve substantial homology with respect to the wild-type substance. This is of particular importance in connection with use of the present variants for treatment in vivo to avoid, or at least reduce, a possible immune response to the variant used for treatment Thus, for pharmaceutical purposes, preferably the present variants are substantially homologous to the corresponding wild-type substance and contain only point mutations, e.g. one or a few single amino acid residue substitutions, deletions and/or insertions. Preferably, the variants contain more than one amino acid residue modification and could contain as many as up to 10 amino acid residue modifications for use in vivo.

Accordingly, suitable variants of PC/APC have a high degree, suitably at least 95%, and preferably at least 97%, and specifically at least 98%, of amino acid sequence identity with wild-type manure PC/APC.

In connection with the diagnostic embodiments of the present invention a high degree of homology is of course of less importance, the main requirement being that the functional variant expresses the desired activity at an enhanced level as compared to the wild-type protein.

For pharmaceutical purposes, preferred embodiments of the present invention are concerned with human PC/APC variants. However, the present invention is also concerned with other PC/APC variants of mammalian origin, e.g. of bovine or murine origin, such as variants of mouse or rat origin, that have enhanced anticoagulant activity due to a modified Gla-domain.

As mentioned above, the Gla-domain or Gla-module is specific for the vitamin K-dependent protein family, the members of which contain a specific protein module (said Gla-module), wherein the glutamic acid A) residues are modified to γ-carboxy glutamic acid residues (Gla). This modification is performed in the liver by enzymes that use vitamin K to carboxylate the side chains of the glutamic acid residues in the protein C precursor. In the sequences (SEQ ID NO 1 and 2), given above for the Gla-domain of human and bovine protein C, respectively, the E residues are thus converted to Gla-residues in the circulating protein.

The Gla-module is comprised of the first amino-terminal 45 residues of the vitamin K-dependent protein and provides the protein with the ability to bind calcium and to bind negatively charged procoagulant phospholipids. Moreover, a membrane contact site, that is of crucial importance for the function of activated protein C (APC) in proteolysis of FVa and FVIIIa, is contained in said Gla-domain, the activity of APC being expressed upon association of APC and other proteins, i.e. factor V and protein S cofactors, on a membrane surface. However, despite a high degree of sequence homology between the Gla-containing regions of different vitamin K-dependent proteins, these proteins display a large range of membrane affinities. This indicates that it could be possible to modify, and more specifically to enhance, membrane affinity of protein C, e.g. human protein C, which is a low affinity protein.

For this purpose, the structures of high affinity vitamin K-dependent proteins could serve as a template to suggest possible modifications that could enhance membrane binding-affinity and, thus, anticoagulant activity of low affinity proteins, such as protein C, as suggested by Shen et al. (supra). For instance, site-directed mutagenesis could be performed on wild-type protein C to produce protein C variants having a structure that approaches the structure of high affinity vitamin K-dependent proteins, such as protein Z.

However, although the existence of a common archetype for electrostatic distribution that would be valid for all vitamin K-dependent proteins and would predict possible positions for amino acid modifications tat could give rise to enhanced membrane binding-affinity is suggested in the WO 99/20767 publication, this archetype is only concerned with a few positions of the Gla-domain, viz. 10, 11, 28, 32, and 33 (according to the numbering used in connection with the present invention). Moreover, as reported by Shen et al. (supra), protein C has been shown to have unique features and would not necessarily fit into such a common hypothesis.

In accordance with the present invention it has unexpectedly been found that modification(s) can be introduced into the Gla-domain of protein C to produce a variant protein C that exhibits improved properties, such as enhanced anticoagulant activity, preferably both in vivo and in vitro. Such variants contain at least one, suitably at least 6, e.g. 7-10, amino acid modification(s), such as substitutions (replacements), deletions or insertions (additions).

According to one aspect of the invention, said at least one amino acid modification is a substitution of one amino acid residue for another residue at a position of the Gla-domain of protein C other than positions 10, 11, 28, 32 or 33. Suitably, said at least one amino acid modification is located at position 12, 23, or 44. A further aspect of the invention is concerned with protein C variants where said at least one amino acid modification is a substitution mutation selected from S12N, D23S and H44Y.

Other embodiments of the present invention are concerned with protein C variants, where said at least one amino acid modification is located at a position selected from positions 10, 11, 12, 23, 32, 33 and 44. Suitably, more than one of, and preferably all, positions 10, 11, 12, 23, 32, 33 and 44 are modified e.g. by substitution.

According to one aspect of the present, said at least one amino acid modification is comprised of one or more amino acid modifications other than E10G11E32D33, Q10G11E32D33, G11N12E32D33, G11E32D33, E32D33, and E32. Alternatively, the present variants could contain one or more of these modifications, provided that this variant contains at least one further modification in the Gla-domain.

A specific human protein C variant having much enhanced anticoagulant activity contains the substitution mutations H10Q, S11G, S12N, D23S, Q32E, N33D and H44Y. Thus, this protein C variant has a modified Gla-domain having the following amino acid sequence:

| | |
|---|---|
| ANSFLEELRQ GNLERECIEE ICSFEEAKEI | (SEQ ID NO:3) |
| FEDVDDTLAF WSKYV | |

It is to be noted that even though variants according to the present invention that contain only the substitutions H10Q, S11G and S12N, only the substitutions D23S, Q32E and N33D, or only the substitutions D23S, Q32E, N33D and H44Y, exhibit a slightly enhanced anticoagulant activity, the above-mentioned specific variant (SEQ ID NO:3) that contains all these substitutions, quite unexpectedly exhibits much enhanced anticoagulant activity as compared to the anticoagulant activity of the protein C variants described by Shen et al. (supra) and in WO 99/20767.

To the man skilled in the art, it is evident that modifications in the Gla-domain other than substitutions could provide protein C variants having improved properties. Moreover, other substitutions than those specifically mentioned herein could also provide such variants. Such substitutions could be conservative or non-conservative. Based on common side chain properties, naturally occurring residues are divided into the following classes:
1) hydrophobic residues comprising norleucine, Met, Ala, Val, Leu and Ile;
2) neutral hydrophilic residues comprising Cys, Ser and Thr;
3) acidic residues comprising Asp and Glu;
4) basic residues comprising Asn, Gln, His, Lys and Arg;
5) residues that influence chain orientation comprising Gly and Pro; and
6) aromatic residues comprising Trp, Tyr and Phe.

Non-conservative substitutions may involve replacement of a member of one of these classes with a member of another class whereas conservative substitutions may involve replacement of an amino acid residue with a member of the same class. Positions of interest for substitutional mutagenesis include positions where the amino acid residues found in wild-type protein C from different species differ, e.g. as regards side-chain bulk, charge, and/or hydrophobicity. However, other positions of interest are such positions where the particular amino acid residue does nor differ between, but are identical for, at least a few different species, since such positions are potentially important for biological activity initially, candidate positions are substituted in a relatively conservative manner. Then, if such substitutions result in a change of biological activity, more substantial substitutions are introduced and/or other modifications, such as additions, deletions or insertions, are made and the resulting variants screened for biological activity.

Since conservative substitutions or modifications of the amino acid sequence could be expected to produce variants having functional and chemical characteristics that are similar to those of wild-type protein C, suitably, the present protein C variants contain at least one non-conservative substitution, e.g. a substitution of an aromatic residue for a basic residue or a basic residue for an acidic residue.

Since the modified, i.e. variant or mutant, PC/APC of the present invention has enhanced anticoagulant activity, the above-mentioned screening for biological activity is suitably concerned with measurement of anticoagulant activity. Such anticoagulant activity can be determined i.a. as the ability of the present variants to increase clotting time in standard in vitro coagulation assays. The enhanced anticoagulant activity is measured in comparison to wild-type PC/APC, which may be derived from plasma or obtained by recombinant DNA technique. Thus, to be useful in accordance with the present invention, the PC/APC variants should express an anticoagulant activity, which is higher than the anticoagulant activity of the wild-type substance. Suitably, the present variants express an anticoagulant activity which is enhanced at least about 400% or more, e.g. up to 1000%, or even up to 3000% over wild-type protein C.

Based on the above and similar principles, a preferred variant of the present invention (SEQ ID NO 3) was constructed. More specifically, in a theoretical paper by MacDonald et al (Biochemistry 1997; 36: 5120-5127) the sequences of all known Gla-domains were compared and an effort was made to correlate the sequences with the abilities of these Gla-domains to bind to negatively charged phospholipid. From this analysis, it was suggested that the great variation in affinities for negatively charged phospholipid among the various Gla domains was related to amino acid sequence differences mainly around residues at position 10 and 32 and 33.

In a previous paper by Shen et al (J biol Chem 1998,273: 31086-31091), several different mutants were created and tested following the theoretical considerations of MacDonald et al. The common theme for these mutants was to change position 11 from a serine (S) to a glycine (G) and position 32 from a glutamine (Q) to a glutamic acid (E, that will be converted to Gla in the mature protein) and position 33 from a asparagine (N) to an aspartic acid (D). In addition, positions 10 and 12 were changed one at the time, but not together. Thus, the mutants tested were E10G11E32D33 (EGED), Q10G11E32D33 (QGED), G11N12E32D33 (GNED) in addition to G11E32D33 (GED), E32D33 (ED) and E32 (E).

It was observed that QGED and GNED were essentially equally effective as anticoagulants and that both were more anticoagulant than wt APC. As compared to wt APC, both mutants bound phospholipid vesicles containing negatively charged phospholipid in a superior manner, and also bound Ca²⁺ more tightly. Even though the most efficient mutants of that study were more anticoagulant than wt APC, this was only found when low concentrations of phospholipid were used. Thus, it was suggested that, even though it was found that improved enzymatic activity of APC correlated with increased membrane affinity for all membranes used, the enhanced affinity of APC for negatively charged phospholipids only improved anticoagulant (enzymatic) activity of APC at low concentrations of negatively charged phospholipids.

Stimulated by the work of Shen et a, (J biol Chem 1998, 273: 31086-31091) the present investigation was initiated. The idea was that possibly more efficient mutations could be created by combining mutations at both positions 10, 11, and 12 into one variant and in addition to test if mutations at positions 23 and 44 could affect the efficiency of the mutant APC. Positions 32 and 33 were believed to be important from the work by Shen et al (J biol Chem 1998, 273: 31086-31091) although it was never proven. The mutants tested by Shen et al. i.e. EGED, QGED, GNED in addition to GED, ED (positions 32, 33) and E position 32) could not prove with certainty the importance of the positions 32 and 33 for the following reasons. The mutants EGED, QGED, GNED and GED were all more efficient than wt APC, but the two mutants ED and E were not more efficient. This raised the possibilities that the mutations around positions 10-12 were those that created the more efficient proteins and that the 32 and 33 mutations were not required. It was hypothesized, but not proven, that the mutations at positions 10-12 had to be combined with mutations at positions 32 and 33. However, it was clear from the Shen et al (J biol Chem 1998,273: 31086-31091) study that mutations at positions 32 and 33 alone were insufficient for the creation of protein C variants exhibiting enhanced anticoagulant activity. As will be demonstrated below, neither mutagenesis at positions 10-12 (the QGN variant) nor at positions 23, 32, 33, and 44 (the SEDY variant) did create molecules with more than slightly improved anticoagulant activity. Only the specific mutant (SEQ ID NO: 3) that contains all the above-identified modifications (designated QGNSEDY or “ALL”) was highly efficient.

As regards amino acid residues suitable for use to substitute wild-type residues at the above-identified positions of wild-type protein C, a comparison of amino acid sequences of different Gla-domains was performed, that included correlation analysis between these amino acid sequence and the phospholipid binding abilities of the different vitamin K-dependent proteins. This suggested that QGN was an interesting option for positions 10, 11, and 12, because both human protein S and bovine factor X comprise these sequences and both these proteins bind negatively charged phospholipid with high affinity. In many Gla domains, position 23 is occupied by a serine (S) residue and that is the reason why the wild-type residue of protein C was replaced with a serine residue when greating a suitable variant of the present invention. It is to be noted that modification of position 44 has not been considered before. However, since the only Gla domain that contains a histidine (H) residue at position 44 is human protein C Gla domain, all other Gla domains having a tyrosine residue at position 44, it seemed logical that replacement of the histidine residue at position 44 with a tyrosine (Y) could be a useful modification.

From the above discussion it is evident that, even though the Gla-domain contains 45 amino acid residues, each of which could be modified independently or in combination, and the APC variant thereby produced would have to be characterized in a search for further variants having enhanced anticoagulant activity, such a search is indeed within reach for the skilled artisan Moreover, based on the state of the art, e.g. using the variants specifically disclosed herein as precursors, fewer variants having essentially the same properties as the precursor variants (e.g. those variants specifically prepared in the experimental part), could be produced, e.g. by introducing one or a few conservative substitutions, or by introducing modifications in parts of the Gla-domain or other parts of the protein C molecule where such modifications would not affect the properties of the precursor that is intended to be modified. Such variants exhibiting essentially unchanged or the same properties as the present variants are considered to be equivalent to the present variants and thus to be encompassed by the present invention.

C. DNA Segments and Preparation Thereof

The present invention is also concerned with the deoxyribonucleic acid (DNA) segments or sequences related to the PC/APC variants, e.g. the structural genes coding for these variants, mutagenizing primers comprising the coding sequence for the modified amino acid stretch, etc.

In this connection, the well-known redundancy of the genetic code must be taken into account. That is, for most of the amino acids used to make proteins, more than one coding nucleotide triplet (codon) can code for or define a particular amino acid residue. Therefore, a number of different nucleotide sequences may code for a particular amino acid residue sequence. However, such nucleotide sequences are considered as functionally equivalent since they can result in the production of the same amino acid residue sequence. Moreover, occasionally, a methylation variant of a purine or pyrimidine may be incorporated into a given nucleotide sequence, but such methylations do not effect the coding relationship in any way. Thus, such functionally equivalent sequences, which may or may not comprise methylation variants, are also encompassed by the present invention.

A suitable DNA segment of the present invention comprises a DNA sequence, that encodes the modified (variant or mutant) PC/APC of the present invention, that is, the DNA segment comprises the structural gene encoding the modified PC/APC. However, a DNA segment of the present invention may consist of a relatively short sequence comprising nucleotide triplets coding for a f w up to about 15 amino acid residues inclusive of the modified amino acid stretch, e.g. for use as mutagenizing primers.

A structural gene of the present invention is preferably free of introns, i.e. the gene consists of an uninterrupted sequence of codons, each codon coding for an amino acid residue present in the said modified PC/APC.

One suitable DNA segment of the present invention encodes an amino acid residue sequence that defines a PC/APC variant that corresponds in sequence to the wild-type human PC/APC except for at least one amino acid modification (insertion, deletion, substitution), in the amino acid sequence corresponding to the Gla-module of the wild-type protein.

Other suitable DNA segments encode PC/APC variants, wherein said modification(s) are contained in the amino acid residue sequence of the Gla-domain at a position other than positions 10, 11, 28, 32, or 33. A preferred DNA-segment encodes a PC variant containing the modifications H10Q, S11G, S12N, D23S, Q32E, N33D and H44Y.

In addition, the present invention is related to homologous and analogous DNA sequences that encode the present PC/APC variants, and to RNA sequences complementary thereto.

The present DNA segments can be used to produce the PC/APC variants, suitably in a conventional expression vector/host cell system as will be explained further below (Section D).

As regards the DNA segments per se, these can be obtained in accordance with well-known technique. For instance, once the nucleotide sequence has been determined using conventional sequencing methods, such as the dideoxy chain termination sequencing method (Sanger et al., 1977), the said segments can be chemically synthesized, suitably in accordance with automated synthesis methods, especially if large DNA segments are to be prepared. Large DNA segments can also be prepared by synthesis of several small oligonucleotides that constitute the present DNA segments followed by hybridization and ligation of the oligonucleotides to form the large DNA segments, well-known methods being used.

If chemical methods are used to synthesize the present DNA segments, it is of course easy to modify the DNA sequence coding for the wild-type PC/APC by replacement, insertion and/or deletion of the appropriate bases encoding one or more amino acid residues in the wild-type molecule.

Suitably, recombinant DNA technique is used to prepare the present DNA segments comprising a modified structural gene. Thus, starting with recombinant DNA molecules comprising a gene, i.e. cDNA encoding wild-type PC/APC, a DNA segment of the present invention comprising a structural gene encoding a modified PC/APC can be obtained by modification of the said recombinant DNA molecule to introduce desired amino acid residue changes, such as substitutions (replacements), deletions and/or insertions (additions), after expression of said modified recombinant DNA molecule. One convenient method for achieving these changes is by site-directed mutagenesis, e.g. performed with PCR-technology. PCR is an abbreviation for Polymerase Chain Reaction, and was first reported by Mullis and Faloona (1987).

Site-specific primer-directed mutagenesis is now standard in the art and is conducted using a synthetic oligonucleotide primer which primer is complementary to a single-stranded phage DNA comprising the DNA to be mutagenized, except for limited mismatching representing the desired mutation(s). Briefly, the synthetic oligonucleotide is used as a primer to direct synthesis of a strand complementary to the phage DNA inclusive of the heterologous DNA and the resulting double-stranded DNA is transformed into a phage-supporting host bacterium. Cultures of the transformed bacteria are plated on top agar, permitting plaque formation from single cells that harbour the phage. In this method, the DNA which is mutated must be available in single-stranded form which can be obtained after cloning in M13 phages. Site-directed mutagenesis can also be accomplished by the “gapped duplex” method (Vandeyar et al., 1988; Raleigh and Wilson, 1986).

In accordance with a suitable embodiment of the present invention, site-directed mutagenesis is performed with standard PCR-technology (Mullis and Faloona, 1987). Examplary PCR based mutagenizing methods are described in the experimental part of the present disclosure. In this example, the replication of the mutant DNA-segment is accomplished in vitro, no cells, neither prokaryotic nor eukaryotic, being used.

Obviously, site-directed mutagenesis can be used as a convenient tool for construction of the present DNA segments that encode PC/APC variants as described herein, by starting, e.g. with a vector containing the cDNA sequence or structural gene that codes and expresses wild-type PC/APC, said vector at least being capable of DNA replication, and mutating selected nucleotides as described herein, to form one or more of the present DNA segments coding for a variant of this invention. Replication of said vector containing mutated DNA may be obtained after transformation of host cells, usually prokaryotic cells, with said vector. Illustrative methods of mutagenesis, replication, expression and screening are described in the experimental part of the present disclosure.

D. Preparation of PC/APC Variants

Such DNA segments, which comprise the complete cDNA sequence or structural gene encoding a PC/APC variant, can be used to produce the encoded variant by expression of the said cDNA in a suitable host cell, preferably a eukaryotic cell. Generally, such preparation of variants of the present invention comprises the steps of providing a DNA segment that encodes a variant of this invention; introduction of the provided DNA segment into an expression vector; introduction of the vector into a compatible host cell; culturing the host cell under conditions required for expression of the said variant; and harvesting the expressed variant from the host cell. For each of the above mentioned steps suitable methods are described in the experimental part of the present disclosure.

Vectors, which can be used in accordance with the present invention comprise DNA replication vectors, which vectors can be operatively linked to a DNA segment of the present invention so as to bring about replication of this DNA segment by virtue of its capacity of autonomous replication, usually in a suitable host cell.

To achieve not only DNA replication but also production of the variant encoded by a DNA segment of the present invention, the said DNA segment is operatively linked to an expression vector, i.e. a vector capable of directing the expression of a DNA segment introduced therein. Replication and expression of DNA can be achieved from the same or different vectors.

The present invention is also directed to recombinant DNA molecules, which contain a DNA segment of the present invention operatively linked to a DNA replication and/or expression vector.

It is well known that the choice of a vector, to which a DNA segment of the present invention can be operatively linked, depends directly on the functional properties desired for the recombinant DNA molecule, e.g. as regards protein expression, and the host cell to be transformed. A variety of vectors commercially available and/or disclosed in prior art literature can be used in connection with the present DNA segments, provided that such vectors are capable of directing the replication of the said DNA segment. In case of a DNA segment containing a structural gene for a PC/APC variant, preferably, the vector is also capable of expressing the structural gene when the vector is operatively linked to said DNA segment or gene.

A suitable embodiment of the present invention is concerned with eukaryotic cell expression systems, suitably vertebrate, e.g. mammalian, cell expression systems. Expression vectors, which can be used in eukaryotic cells are well brown in the art and are available from several commercial sources. Generally, such vectors contain convenient restriction sites for insertion of the desired DNA segment. Typical of such vectors are pSVL and pKSV-10 (Pharmacia), pBPV1/pML2d (International Biotechnologies, Inc.), pXT1 available from Stratagene (La Jolla, Calif.), pJ5Eω available from The American Type Culture Collection (ATCC; Rockwille, Md.) as accession number ATCC 37722, pTDT1 (ATCC 31255) and the like eukaryotic expression vectors. In the experimental part of the present disclosure, pRc/CMV (available from Invitrogen, California, U.S.A.) has been used to obtain expression plasmids for use in adenovirus-transfected human kidney cells.

Suitable eukaryotic cell expression vectors used to construct the recombinant DNA molecules of the present invention include a selection marker that is effective in eukaryotic cells, preferably a drug resistance selection marker. A suitable drug resistance marker is the gene whose expression results in neomycin resistance, i.e. the neomycin phosphotransferase (neo) gene, Southern et al., J. Mol. Appl. Gene., 1:327-341 (1932). A further suitable drug resistance marker is a marker giving rise to resistance to Geneticin (G418). Alternatively, the selectable marker can be present on a separate plasmid, in which case the two vectors will be introduced by co-transfection of the host cell and selection is achieved by culturing in the appropriate drug for the selectable marker.

Eukaryotic cells, which can be used as host cells to be transformed with a recombinant DNA molecule of the present invention, are not limited in any way provided tat a cell line is used, which is compatible with cell culture methods, methods for propagation of the expression vector and methods for expression of the contemplated gene product. Suitable host cells include yeast and animal cells. Vertebrate cells, and especially mammalian cells are preferred, e.g. monkey, murine, hamster or human cell lines. Suitable eukaryotic host cells include Chinese hamster ovary (CHO) cells available from the ATCC as CCL61, NIH Swiss mouse embryo cells NIH/3T3 available from the ATCC as CRL1658, baby hamster kidney cells (BHK) and the like eukaryotic tissue culture cell lines. In the experimental part of the disclosure, an adenovirus-transfected human kidney cell line 293 (available from American Type Culture Collection, Rockville, Md., U.S.A.) has been used.

To obtain an expression system in accordance with the present invention, a suitable host cell, such as a eukaryotic, preferably mammalian, host cell, is transformed with the present recombinant DNA molecule, known methods being used, e.g. such methods as disclosed in Graham et al., Virol., 52:456 (1973); Wigler et al., Proc. Natl. Acad. Sci. USA, 76:1373-76 (1979).

Thus, to express the DNA segment of the present invention in a eukaryotic host cell, generally, a recombinant DNA molecule of the present invention is used that contains functional sequences for controlling gene expression, such as an origin of replication, a promoter which is to be located upstream of the DNA segment of the present invention, a ribosome-binding site, a polyadenylation site and a transcription termination sequence. Such functional sequences to be used for expressing the DNA segment of the present invention in a eukaryotic cell my be obtained from a virus or viral substance, or may be inherently contained in the present DNA segment, e.g. when said segment comprises a complete structural gene.

A promoter that can be used in a eukaryotic expression system may, thus, be obtained from a virus, such as adeno-virus 2, polyoma virus, simian virus 40 (SV40) and the like. Especially, the major late promoter of adenovirus 2 and the early promoter and late promoter of SV40 are preferred.

A suitable origin of replication may also be derived from a virus such as adenovirus, polyoma virus, SV40, vesicular stomatitis virus (VSV) and bovine papilloma virus (BPV). Alternatively, if a vector, that can be integrated into a host chromosome, is used as an expression vector, the origin of replication of the host chromosome may be utilized.

Even if eukaryotic expression systems are preferred, prokaryotic expression systems can also be used in connection with the present invention. Moreover, prokaryotic systems can advantageously be used to accomplish replication or amplification of the DNA-segment of the present invention, subsequently the DNA segments produced in said prokaryotic system being used for expression of the encoded product, e.g. in a eukaryotic expression system.

Thus, a prokaryotic vector of the present invention includes a prokaryotic replicon, i.e. a DNA sequence having the ability to direct autonomous replication and maintenance of the recombinant DNA molecule extrachromosomally in a prokaryotic host cell, such as a bacterial host cell, transformed therewith. Such replicons are well known in the art. In addition, those embodiments that include a prokaryotic replicon also include a gene, whose expression confers drug resistance to a bacterial host transformed therewith. Typical bacterial drug resistance genes are those tat confer resistance to ampicillin or tetracycline.

If a prokaryotic system is used, not only for DNA replication but also as an expression system, these vectors that include a prokaryotic replicon also include a prokaryotic promoter capable of directing the expression, i.e. transcription and translation, of the present DNA segment containing a structural gene, in a bacterial host cell, such as E. coli, transformed therewith. A promoter is an expression control element formed by a DNA sequence that permits binding of RNA polymerase and transcription to occur.

Promoter sequences compatible with bacterial hosts are typically provided in plasmid vectors containing convenient restriction sites for insertion of a DNA segment of the present invention. Typical of such vector plasmids are pUC8, pUC9, pUC18, pBR322 and pBR329 available from BioRad Laboratories, Richmond, Calif. and pPL and pKK223 available from Pharmacia.

Accordingly, to obtain a prokaryotic expression system which can express the gene product of the present invention appropriate prokaryotic host cells are transformed with a recombinant DNA molecule of the present invention in accordance with well known methods that typically depend on the type of vector used, e.g. as disclosed in Maniatis et al., Molecular Cloning, A Laboratory Manual, Cold Spring Harbor Laboratory, Cold Spring Harbor, N.Y. (1982).

It is of course necessary that successfully transformed prokaryotic or eukaryotic cells can be distinguished and separated from non-transformed cells. For this purpose, a variety of methods are known and have been described in prior art literature.

In accordance with one such method, the presence of recombinant DNA is assayed for by examining the DNA content of monoclonal colonies derived from cells which have been subjected to a transformation procedure. Such methods have been disclosed by Southern, J. Mol. Biol. 98:503 (1975) and Berent et al., Biotech., 3:208 (1985).

Successful transformation can also be confirmed by well-known immunological methods, e.g. using monoclonal or polyclonal antibodies specific for the expressed gene product, or by the detection of the biological activity of the expressed gene product.

Thus, cells successfully transformed with an expression vector can be identified by the antigenicity or biological activity that is displayed. For this purpose, samples of cells suspected of being transformed are harvested and assayed for either the said biological activity or antigenicity.

Such selected, successfully transformed cells are used to produce the desired PC/APC variants as disclosed above.

E. Assays for Biological Activity

Suitable methods for assaying the biological activity of the PC/APC variants of the present invention are based on plasma clotting systems, such as an APTT system, and on tests related to degradation of purified Factor VIIIa. Such methods are disclosed in more detail in the experimental part of the present disclosure.

F. Compositions

The present PC/APC variants are typically provided in a compositional form that is suitable for the intended use. Such compositions should preserve biological activity of the PC/APC variant and also afford stability thereof. Suitable compositions are therapeutic compositions that contain a therapeutically active amount of a variant according to the present invention, e.g. in combination with a physiologically tolerable carrier. Such compositions could also contain a therapeutically active amount of a further active ingredient, such as protein S and/or Factor V, to enhance the anticoagulant activity thereof. Since protein C is a calcium dependent protein, suitably, the present compositions also contain divalent calcium, preferably in a physiological amount.

Since considerations to be taken into account in connection with design of compositional forms in general, and specifically therapeutic compositions, are well known to the skilled artisan, there is no need to describe these in more detail.

G. Therapeutic Methods

According to the present invention, it has been shown that the present PC/APC variants exhibit an enhanced anticoagulant activity. Thus, the present invention is also concerned with methods for inhibiting coagulation in an individual, e.g. a human, said method comprising administering to said individual a composition comprising a therapeutically effective amount of a variant PC/APC of the present invention. Conditions that could be treated are disclosed elsewhere in this specification.

As for compositions, considerations to be taken into account in connection with design of therapeutic methods, e.g. suitable dosage ranges and administration routes, are well known to the skilled artisan, and, thus, there is no need to describe these methods in more detail.

H. Discussion

A specific variant according to the present invention that has been prepared in the experimental part and is designated QGNSEDY (ALL) has been found to exhibit much improved properties.

This variant is more anticoagulant than wt APC and it is also more active than previous mutants such as GNED or QGED (described by Shen et al, supra). It is quite a surprise that this variant exhibits a much enhanced activity, i.a. since neither of the two variants QGN and SEDY exhibits any, or only exhibits a slightly, increased anticoagulant activity or increased affinity for negatively charged phospholipid membranes. This suggests that the membrane binding ability of the Gla-domain is very complex and not easily affected by single amino acid replacements. Only when multiple areas of the Gla-domain are mutated, it is possible to obtain a unique variant like QGNSEDY (ALL) that exhibits much enhanced phospholipid affinity and much increased anticoagulant activity.

The anticoagulant activity of QGNSEDY (ALL) is potentiated by protein S, which stands in contrast to the activity of a chimeric APC variant described by Smirnov and Esmon in U.S. Pat. No. 5,837,843. This variant is a hybrid between protein C and prothrombin, wherein the prothrombin Gla-domain is replacing the corresponding Gla-domain in protein C (PC). Although, due to enhanced phospholipid binding, this PC/APC variant is more anticoagulant than wild-type APC, its activity is not potentiated by protein S.

Also EP 0 296 413 A2 is concerned with protein C hybrids, not only between prothrombin and PC but also between FVII, FIX, or FX and PC. These variants contain the Gla domain from prothrombin, FVII, FIX, or FX and the rest from PC. However, in these variants the Gla-domain has been limited to the first N-terminal 43 amino acid residues and thus, these variants do not contain a modified amino acid residue at position 44 of wt protein C. Although it is stated therein, that these variants have improved activity against blood clot formation or improved fibrinolysis accelerating effect, these variants have not been well characterized as regards such activities. Only a FX/PC hybrid has been prepared and characterized and this hybrid was not found to have improved anticoagulant properties over wt PC apart from improved inactivation of factor Va.

A further quite unexpected advantage with the present variant QGNSEDY (ALL) is that it is able to cleave FVa that is mutated at its main cleavage site by APC, i.e. position Arg506 (designated. FV:Q506 or FV Leiden) and is present in the common blood coagulation disorder designated APC resistance. This is an advantage over wild-type APC that is very poor in cleaving the Arg306, which is the site that when cleaved results in complete inactivation of FVa. Thus, contrary to wild-type APC, the present variant QGNSEDY (ALL) is capable of cleaving and inactivating activated FV:Q506. In contrast to the cleavage at Arg506, the cleavage at Arg306 is potentiated by protein S. However, a further advantage of the present variant QGNSEDY (ALL) is that it cleaves activated FV:Q506 even in absence of protein S. Yet, this cleavage is stimulated by protein S, even though protein S is not required. The ability of the present variant QGNSEDY to cleave activated factor V at Arg306, makes it attractive as an anticoagulant also for patients with APC resistance.

It is obvious that a recombinant protein C molecule which after its activation to APC expresses enhanced anticoagulant activity has great potential use both as a possible therapeutic compound and as a reagent to be used in various biological assays for other components of the protein C system. In accordance with the present invention it has been shown that mutations in the Gla module of the protein C molecule can lead to substantially enhanced anticoagulant activity, mainly due to enhanced membrane-binding activity. Thus, it can be expected that a systematic search for such mutations may produce other protein C molecules with even better properties. For instance, it could become possible to design APC molecules with highly specific functions, e.g. further molecules that cleave FVa at Arg306 and thus to produce fierier APC variants which works well against said mutated FV which is present in the blood coagulation disorder APC-resistance.

It is envisioned that the present protein C variants expressing enhanced anticoagulant activity will be useful in all situations where undesired blood coagulation is to be inhibited. Thus, the present variants could be used for prevention or treatment of thrombosis and other thromboembolic conditions. Illustrative of such conditions are disseminated intravascular coagulation (DIC), arteriosclerosis, myocardial infarction, various hypercoagulable states and thromboembolism and also sepsis and septicaemia. The present variants could also be used for thrombosis prophylaxis, e.g. after thrombolytic therapy in connection with myocardial infarction and in connection with surgery. A combination of the present protein C variants and protein S (wild-type protein S or a variant thereof) could be useful, which combination also could include Factor V expressing activity as a cofactor to APC.

As regards diagnostic use of the present PC/APC variants, there is a great need for improved functional assays for protein S and also for the anticoagulant activity of factor V. It is likely that a mutated APC wit enhanced anticoagulant activity will be very useful in such assays because such APCs will give stronger signal and this will lead to increased signal to noise ratios in different assays.

It might be possible to combine mutations in the Gla-module with mutations in other parts of protein C to produce protein C with very unique properties. The scientist at Ely Lilly (Ehrlich et al, Embo. J. 1990, 9:2367-2373; Richardson et al, Nature 1992,360:261-264) and also other groups have already shown that mutations around the activation peptide region yielded protein C which was easily activated even in the absence of TM. Similarly, another set of mutations in the activation peptide region led to a protein C molecule which was secreted in active form from the synthesizing cells (Ehrlich et at, J. Biol, Chem. 1989,264:14298-14304). In a future perspective is may become interesting to combine mutations affecting the activation process and/or mutations in the SP-module which affect the catalytic activity, with the present mutations in the Gla-domain. Also combinations of the present mutations with future mutations that may enhance the interactions between APC and its cofactors, are envisioned.

### Experimental Part

In the following examples suitable embodiments are disclosed that illustrate the present invention. However, these examples should not be construed as limiting the invention. Unless otherwise stated therein, human PC/APC variants have been prepared and human coagulation factors, plasma, etc. have been used.

In these examples, the following materials were used.

Lipofectin and Geneticin (G418) are available from Life Technologies AB, Sweden, and Dulbecco's Eagle's modified medium (DMEM) is available from Gibco Corp.

Thrombin and human protein S were purified according to previously described methods (Dahlbäck, et al, 1990; Dahlbäck and Hildebrand, 1994).

### EXAMPLE 1

### Preparation of Variants of Protein C

(a) Site Directed Mutagenesis

The various protein C variants used in this study were created with recombinant technologies essentially as described previously by Shen et al (J Biol Chem 1998,273: 31086-31091 and in Biochemistry 1977, 36 16025-16031).

A full-length human protein C cDNA clone, which was a generous gift from Dr. Johan Stenflo (Dept. of Clinical Chemistry, University Hospital, Malmö, Sweden), was digested with the restriction enzymes HindIII and XbaI and the resultant restriction fragment comprising the complete PC coding region, that is full length protein C cDNA, was cloned into a HindIII and Xbal digested expression vector pRc/CMV.

The resultant expression vector containing the coding sequence for wild-type human protein C was used for site-directed mutagenesis of the Gla-module of protein C, wherein a PCR procedure for amplification of target DNA was performed as described previously (Shen et al., supra).

Mutagenesis primers were designed for use in this procedure to cause replacement of the wild-type amino acid residues at positions 10, 11, 12, 23, 32, 33, and 44 with various other amino acids. More specifically, at position 10, histidine (H) was replaced with glutamine (Q); at position 11, serine (S) was replaced with glycine (G); at position 12, serine was replaced with asparagine (N); at position 23, aspartic acid (D) was replaced with serine (S); at position 32, glutamine (Q) was replaced with glutamic acid (E), which in the mature protein will be converted to a Gla (gamma-carboxy glutamic acid); at position 33, asparagine (N) was replaced with an aspartic acid (D); and finally at position 44, histidine (H) was replaced with a tyrosine (Y). These primers were used to produce the following variants (or mutants):
Mutant 1) designated QGN (positions 10, 11, 12 were mutated).
Mutant 2) designated SED (positions 23, 32, and 33 were mutated).
Mutant 3) designated SEDY (positions 23, 32, 33, and 44 were mutated).
Mutant 4) designated QGNSEDY, which is a combination of mutants 1) and 3) (QGN and SEDY).
Mutant 5) designated GNED and mutant 6) designated QGED (both previously described by Shen et al) were used as comparison.

To create the QGN mutant, the two following oligonucleotides were synthesized and used in the first PCR procedure, viz. primer A having the nucleotide sequence: 5′-AAA TTA ATA CGA CTC ACT ATA GGG AGA CCC AAG CTT-3′ (SEQ ID NO:4) (corresponding to sense of nucleotides 860-895 in the vector pRc/CMV including the HindIII cloning site) and primer B having the nucleotide sequence: GCA CTC CCG CTC CAG GTT GCC TTG ACG GAG CTC CTC CAG GAA (SEQ ID NO:5) (corresponds to the second strand of the DNA stretch that encodes amino acids 4-17 with positions 10-12 mutated, which is shown by the underlining of the corresponding nucleotides). These primers A and B were used in the PCR reaction wherein wt human protein C cDNA was used as template. The PCR product was cleaved with HindIII and Bsr BI that yielded an approximately 200 bp long fragment containing the mutant amino acid residues. This fragment was ligated to two other DNA pieces, one being a Bsr BI-Xba I fragment encoding a large part of wt human protein C cDNA and the other being the Hind III-Xba I cleaved pRc/CMV vector. The ligated cDNA was checked with restriction enzyme cleavage (Hind III/Bsr BI) and sequencing to confirm the QGN mutations.

Several steps were made to create the SEDY. The first was to create the S23 mutation in a cDNA that had already the E32D33 mutation (Shen et al J Biol Chem 1998, 273: 31086-31091). Two primers were made for the S23 mutation, one being designated primer C and the other being designated primer D. Primer C had the nucleotide sequence: ATA GAG GAG ATC TGT AGC TTC GAG GAG GCC AAG (SEQ ID:6) (mutation is underlined); and primer D had the nucleotide sequence: CTT GGC CTC CTC GAA GCT ACA GAT CTC CTC TAT (SEQ ID NO:7) (mutation is underlined). To create mutant cDNA, two PCR reactions were performed wherein mutant cDNA ED was used as a template and wherein primers A and C were used in the first reaction whereas primers D and E were used in the second reaction. Primer E had the nucleotide sequence: 5′-GCA TTT AGG TGA CAC TAT AGA ATA GGG CCC TCT AGA-3′ (SEQ ID NO:8) (antisense to nucleotides 984-1019 in the vector pRc/CMV including the Xba I cloning site). The first PCR reaction that involved primers A and C amplified the 5′ part of the protein C cDNA (encoding up to amino acid 28), whereas the second PCR reaction that involved primers D and E generated the 3′ part of the cDNA encoding from amino acid 18 until the end of the protein C. The two products produced in these reactions were ten combined in a further PCR reaction wherein primers A and E were used. The final product from this procedure was a cDNA encoding the whole protein C carrying mutations at positions 23, 32 and 33. Then, the PCR product was cleaved with Hind III and Sal I which gave a 360 bp 5′ fragment that was purified and ligated with the Sal I-Xba I fragment of wt protein C into the Hind III-Xba I cleaved pRc/CMV vector. This vector thus contained cDNA for the full-length mutant SED. This cDNA was used as template in a PCR reaction to create the mutant SEDY, i.e. position 44 was mutated from histidine to a tyrosine (Y). In is reaction, primer A was combined with a primer F designed to mutate position 44 and having the following nucleotide sequence: CTG GTC ACC GTC GAC GTA CTT GGA CCA GAA GGC CAG (SEQ ID NO:9) (corresponds to the second strand encoding amino acid residues 39-49—the underlined codon being the mutation spot). The PCR product was cleaved with Hind III and Sal I and the about 360 bp long fragment was ligated to the remaining part of the protein C cDNA, i.e. the Sal I-Xba I fragment and the Hind III -Xba I cleaved pRc/CMV.

The fully mutated protein C cDNA, that encodes the mutant QGNSEDY, was then created using cDNAs for the QGN and SEDY mutants. The combination was created using restriction enzyme digestion and ligation of appropriate fragments. Thus, the QGN variant cDNA was cleaved with Hind III and Bsr BI and the about 200 bp long 5′ fragment was isolated and used together with the Bsr BI-Xba I fragment (about 1000 bp long) derived from the SEDY cDNA. The two fragments were ligated into Hind III-Xba I cleaved pRc/CVM to generate the full length variant protein C cDNA encoding QGNSEDY (also referred to as “ALL” in this text). The final product was tested with sequencing and found to contain the correct mutations.

For the record, the E32D33 mutant was created in a similar fashion (this mutant is described in detail in Shen et al J Biol Chem 1998, 273: 31086-31091) using the primer G: 5′-CAG TGT GTC ATC CAC ATC TTC GAA AAT TTC CTT GGC-3′ (SEQ ID NO:10) (antisense for amino acids 27-38 with the E32D33 mutation underlined).

DNA sequencing confirmed all mutations. Cell culture in human 293 cells, expression, purification, and characterization on of protein C molecules were performed as described (Shen, L et al J Biol Chem 1998, 273: 31086-31091).

In brief, the resultant human protein C cDNA containing the desired mutations was digested with SacII and ApaI, and then the fragment from the SacII and ApaI digestion (nucleotides 728-1311) was cloned into the vector pUC 18 which contains intact human protein C fragments (HindIII-SacII, 5′end-nucleotide 728; and ApaI-XbaI, nucleotide 1311-3′ end) to produce human protein C full length cDNA comprising the desired mutations, viz. coding for a human protein C mutant comprising the mutated sequence instead of the human wild-type sequence.

Then, each of the above mutated human protein C cDNAs was digested with HindIII and XbaI and the appropriate restriction fragment was cloned into the vector pRc/CMV, which had been digested with the same restriction enzymes. The vectors obtained were used for expression of mutated human protein C in eukaryotic cells.

Before transfection of the appropriate host cells, all mutations were confirmed by DNA sequencing by the dideoxy chain termination method of Sanger et al., supra

(b) Production of Stable Transformants Producing Variant or Wild-type Protein C

To produce stable transformants producing variant or wild-type protein C, adenovirus-transfected human kidney cell line 293, was grown in DMEM medium containing 10% of fetal calf serum 2 mM L-glutamine, 100 U/ml penicillin, 100 U/ml streptomycin and 10 μg/ml vitamin K₁, and transfected with an expression vector comprising wild-type or mutagenized protein C cDNA from step (a). The transfection was performed in accordance with the Lipofectin method as described earlier (Felgner et al., 1987). In brief, 2 μg of vector DNA which was diluted to 100 μl with DMEM containing 2 MM of L-glutamine was mixed with 10 μl Lipofectin (1 μg/μl) which was diluted to 100 μl with the same buffer. The mixture was kept at room temperature for 10-15 min and was diluted to 1.8 ml with the medium, and then added to the cells (25-50% confluence in a 5-cm Petri dish) that had been washed twice with the same medium.

(c) Expression of Variant or Wild-type Protein C

The transfected cells from (b) were incubated for 16 hours, whereafter the medium was replaced with complete medium containing 10% calf serum and the cells were incubated for additional 48-72 hrs. The cells were then trypsinized and seeded into 10-cm dishes containing selection medium (DMEM comprising 10% serum, 400 μg/ml G418, 2 mM L-glutamine, 100 U/ml penicillin 100 U/ml streptomycin and 10 μg/ml vitamin K₁) (Grinnell, et al. 1990). G418-resistant colonies were obtained after 3-5 weeks selection. From each DNA transfection procedure, 24 colonies were selected and grown until confluence. All colonies were screened by dot-blot assays using monoclonal antibody HPC₄ (specific for human protein C) to examine the protein C expression. High expression cell colonies were selected and grown until confluence in the selection medium. Thereafter, these cells were grown in a condition medium (selection medium lacking serum) to initiate expression of protein C or a variant thereof, which medium, like the selection medium was replaced every 72 h. After a suitable time period, the condition medium containing the respective expression product was collected for purification of said product in section (d) below.

(d) Purification of Recombinant Wild-type and Mutated Proteins

Culture medium obtained in section (c) from transformants producing human wild-type or mutant protein C was subjected to a simple and convenient purification method comprising a chromatographic method termed “pseudo- affinity” and described earlier (Yan et al., Biotechnology 1990, Vol. 8, 665-61).

The purified proteins obtained above were concentrated on YM 10 filters (Amicon), dialyzed against TBS buffer (50 mM Tris-HCl and 150 mM NaCl, pH 7.4) for 12 hrs and stored at −80° C. until use thereof.

The purity and homogeneity of the above wild-type and mutant protein C's were established by SDS-PAGE. This electrophoresis procedure was run as a polyacrylamide (10-15%) slab-gel electrophoresis in the presence of 0.1% of SDS (sodium dodecyl sulphate) under reducing and non-reducing conditions wherein the said proteins were visualized by silver staining (Morrissey, 1981).

### EXAMPLE 2

### Characterization of Protein C Mutants

To characterize the protein C mutants obtained in the previous steps, mutant and wild-type protein C's were activated and their anticoagulant activity was tested in different experimental systems, including plasma-based assays and set ups with purified components.

Two plasma systems were tested, one being the activated partial thromboplastin time (APTT) system and the other being the thromboplastin (TP) system. In both the APTT and the TP systems, the anticoagulant activity of increasing concentrations of wt or mutant APCs was tested. In the APTT system, the anticoagulant activity of APC is dependent both on FVIIIa and FVa degradation, whereas the TP system is mainly sensitive to FVa degradation. However, the diluted TP system is to some extent sensitive also to degradation of FVIIIa

(a). Inhibition of Clotting by APC Variants as Monitored by an APTT Reaction

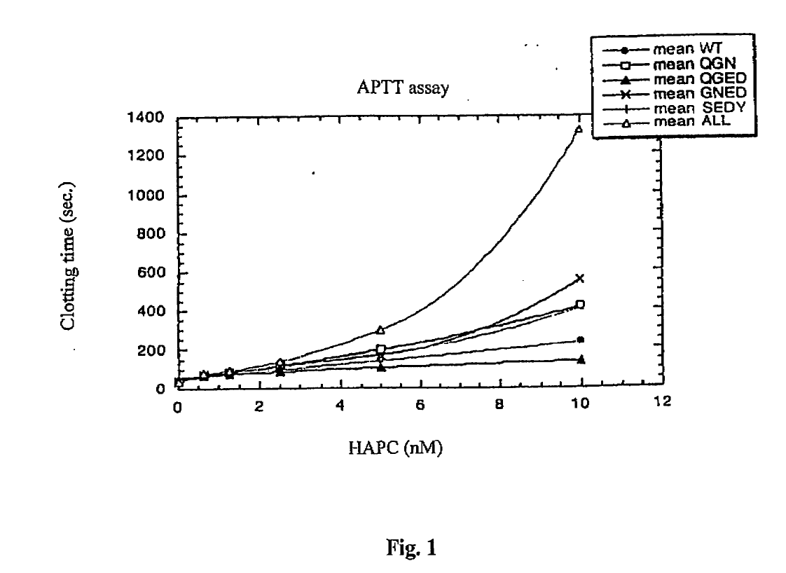

(i) Method: Plasma (50 μl) was mixed with 50 μl APTT reagent (APTT Platelin LS from Organon Technica) and incubated for 200 seconds at 37° C. Coagulation was initiated with a mixture of 50 μl APC (final concentration given in FIG. 1) and 50 μl 25 mM CaCl_{2.} The clotting time was measured in an Amelung coagulometer.

(ii) Results: In this APTT-based assay, the activity of wt APC was compared with the activity of the mutants 1), 3), and 4), i.e. QGN, SEDY, and QGNSEDY (ALL), of the present invention, as well as with the activity of two mutants previously described by Shen et al (J biol Chem 1998, 273: 31086-31091), i.e. mutants 5) and 6) designated GNED and QGED, respectively.

With reference to FIG. 1, it is evident that the anticoagulant activity of ALL is considerably enhanced in comparison to the anticoagulant activity of wt APC. At the highest concentration used, ALL yielded clotting times exceeding 1000 seconds, whereas wt APC only gave a clotting time of about 200 seconds. The basal normal clotting time without added APC is about 30-45 seconds. On the other hand, the two previously described mutants, QGED and GNED gave very different results. OWED was considerably more active than wt APC, whereas QGED in fact was less active than wt APC. The variants QGN and SEDY of the present invention were equally active as GNED but were less active than ALL.

In this APTT assay, the reagents were standard commercial reagents, which stands in contrast to the reagents used in the study by Shen et al. (J biol Chem 1998, 273: 31086-31091) In that study, a diluted APTT regent was used, since otherwise the APC variants were not more active anticoagulants than wt APC. In the discussion section of the Shen et al reference, this was explained to be due to the level of phospholipid in the reagents. If high levels of phospholipid were used, it was not easy to notice the increased activity of the APC variants used in the study by Shen et al. Only when diluted regents were used, the authors could demonstrate a strong increase in the anticoagulant activity of the APC variants.

The present variant QGNSEDY (ALL) appears to be unique as it is evidently much more active than wt APC, also at standard levels of phospholipid.

(b) Impact of Human Protein S in an APTT Assay

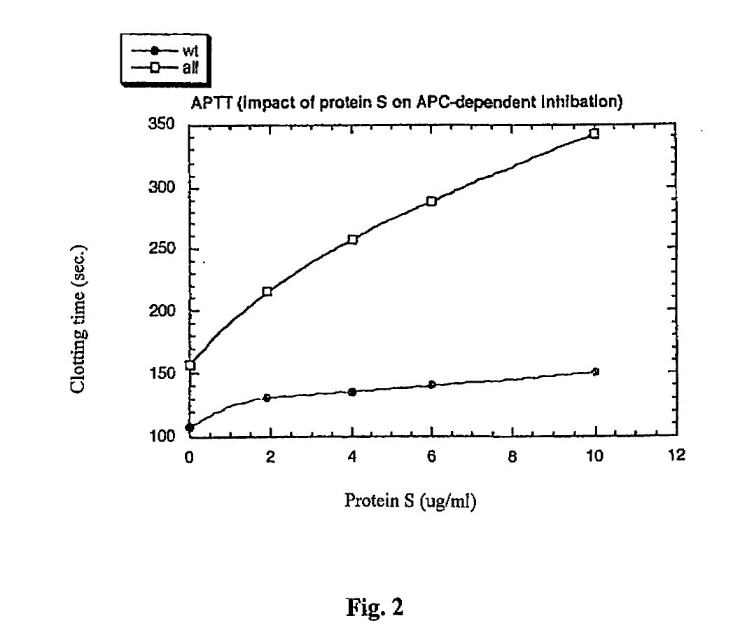

(i) Method; Increasing concentrations of protein S were added to protein S deficient plasma to obtain the final concentrations indicated in FIG. 2. Plasma aliquots (50 μl) were mixed with the APTT reagent and then incubated for 200 seconds at 37° C. APC, either wt or the ALL mutant (QGNSEDY), was added in a volume of 50 μl (concentration 20 nM and clotting was then immediately initiated by the addition of 50 μl of 25 mM CaCl_{2.} The results are shown in FIG. 2, as clotting times plotted versus the concentration of protein S in the protein S deficient plasma.

These experiments were performed essentially as described above with reference to FIG. 1, protein S deficient plasma being used instead of the normal plasma. This protein S deficient plasma was of human origin and the protein S depletion was the result of immune-absorption using a highly efficient monoclonal antibody against human protein S (HPS54-described by Dahlbäck et al. (J Biol Chem 1990 265: 8127-35).

(ii) Results: With reference to FIG. 2, it is evident that a preferred variant of the present invention, viz. the QGNSEDY valiant, was considerably more active than wt APC also when protein S depleted plasma was used. Of particular interest is the observation, that the addition of exogenous protein S enhanced the anticoagulant activity of QGNSEDY as well as of wt APC. In absence of protein S, the mutant ALL yielded a clotting time of about 160 seconds and this clotting time was prolonged up to 350 seconds by the addition of protein S in the test system. Corresponding values obtained with wt APC were a basal clotting time of about 100 seconds in the absence of protein S and a prolonged clotting time of 150 seconds in the presence of the highest protein S concentration used in this test. Thus, it is obvious that ALL is essentially more active than wt APC both in presence and absence of protein S and that ALL moreover is potentiated by the presence of protein S. This is in contrast to the results obtained by Esmon and Smirnov with their APC variants (described in WO 98/20118) that were not stimulated by protein S. Evidently, the present variant QGNSEDY is superior to the variants disclosed by Esmon and Smirnov, since it is stimulated by protein S.

(c) Inhibition of Clotting by APC Variants as Monitored by a TP System

(i) Method: Normal plasma (50 μl) was mixed with increasing concentrations of the various APC variants (50 μl aliquots whereafter clotting was initiated by the addition of thromboplastin diluted 1/50 as a source of tissue factor. To initiate clotting, the diluted thromboplastin also contained 25 mM CaCl_{2.}

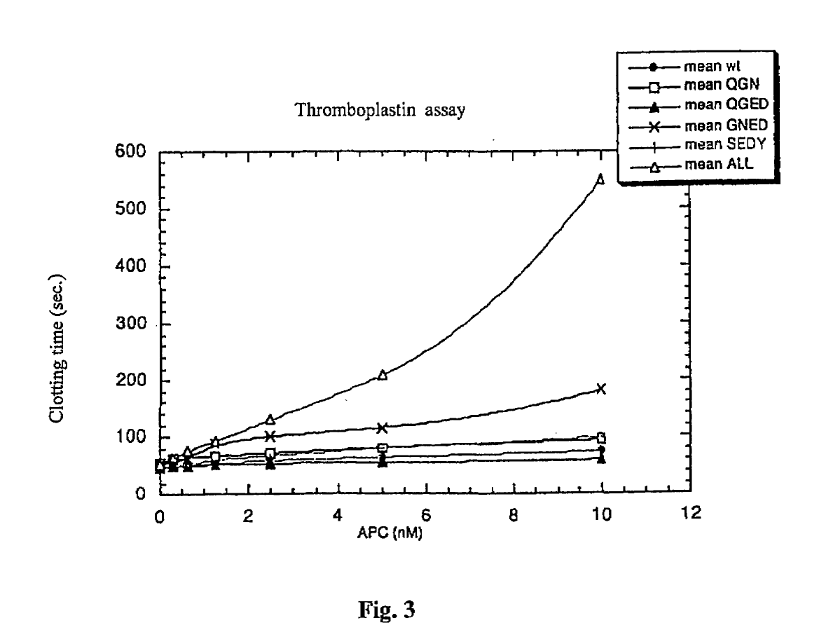

(ii) Results: As is evident from FIG. 3, the results obtained with this assay were similar to those obtained with the APTT system. Thus, the variant QGNSEDY was considerably more active than wt APC. More specifically, at the highest concentration used, the variant QGNSEDY (designated ALL in FIG. 3) yielded a clotting time that was close to 600 seconds. The second best variant was GNED, which at the highest concentration yielded a clotting time of approximately 180 seconds. In contrast, wt APC only yielded clotting times of about 70 seconds. The basal clotting time obtained without addition of exogenous APC was approximately 40 seconds.

Apparently, the results of this experiment suggest that as compared to wt APC the variant QGNSEDY has unique properties, since wt APC never exhibits an anticoagulant activity as high as the anticoagulant activity of the variant QGNSEDY, not even at increasing concentrations of wt APC. This might suggest tat by mutagenesis of the Gla-domain of the variant QGNSEDY, a molecule has been created that exhibits new and distinct functions as compared to wt APC. One such function could be related to the protection of the Arg506 site in FVa that is provided by FXa. It is known that FXa binds to FVa at a site close to Arg506 and that this results in protection of the Arg506 site. Possibly, the unique and high phospholipid binding ability of QGNSEDY abrogates the protection provided by FXa. During the clotting assays, a certain amount of FXa is formed and this may restrict the ability of wt APC to cleave the Arg506 site in FVa. It is possible that the QGNSEDY variant could replace the FXa due to its high affinity not only for phospholipid membranes but also for the FVa molecule. Moreover, at the highest concentration of APC used in this test, the QGNSEDY variant is able to prolong the clotting times considerably more than wt APC is able to. This suggests that the APC variant QGNSEDY might have unique in vivo properties and may be able to inhibit a clotting reaction that is already ongoing.

(d) Impact of Protein S in a PT Assay

Experiments with protein S deficient plasma like those described in Example 2(b)(i), were also performed, the thromboplastin system of Example 2(c)(i) being used. The results thereby obtained were similar to those described for the APTT system in Example 2(b)(ii).In brief, it was found that the QGNSEDY variant is active in the absence of protein S, but yet, its activity is potentiated by protein S.

### EXAMPLE 3

### Inactivation of FVa by APC

In this example, the enhanced activity of the APC variant QGNSEDY was established in a system, designed to more specifically characterize the degradation of FVa and wherein the loss of FVa activity over time is demonstrated.

(i) Method: Plasma FVa (0.76 nM) (plasma was diluted 1/25 and FV contained therein was activated by the addition of thrombin—this was used as the source of FVa) was incubated with APC (0.39 nM) in the presence of 25 μM phospholipid vesicles (mixture of 10% phosphatidylserine and 90% phosphatidylcholine). The buffer was 25 mM Hepes, 0.15 M NaCl, 5 mM CaCl, pH 7.5, and 5 mg/ml BSA and the temperature was 37° C.

At various tine points, aliquots were drawn and the remaining FVa activity was determined by a FVa assay. This assay was based on the ability of FVa to potentiate the FXa-mediated activation of prothrombin. This assay contained bovine FXa (5 nM final concentration), 50 μM phospholipid vesicles (mixture of 10% phosphatidylserine and 90% phosphatidylcholine) and 0.5 μM bovine prothrombin. The generation of thrombin was measured using the chromogenic substrate S2238 (available from Chromogen AB).

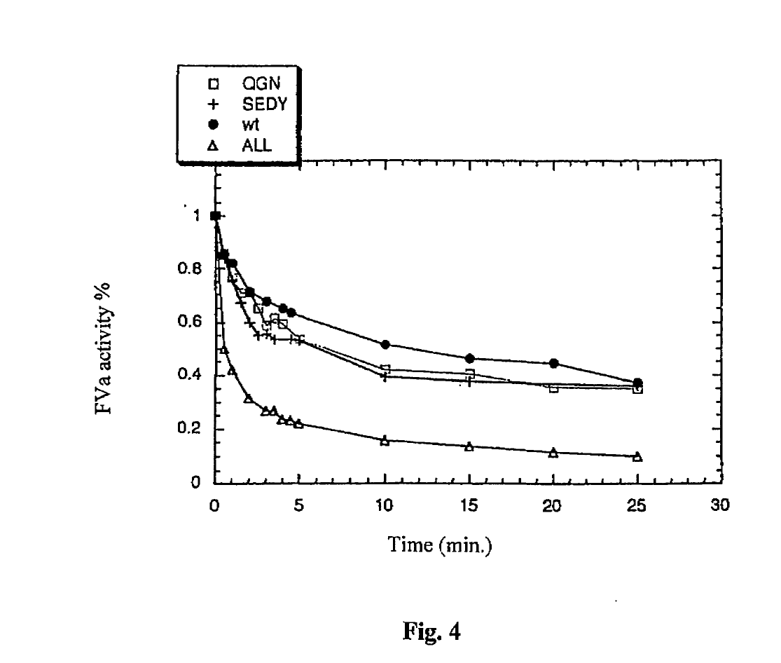

(ii) Results: The loss of FVa activity that follows upon incubation of FVa with wt APC is the result of primarily two cleavage reactions, viz. at Arg506 and at Arg306. The kinetically favored reaction is the reaction occuring at Arg506, tat yields the initial rapid loss of FVa activity that is observed during the first 5 minutes of incubation. The Arg506 cleavage only results in partial inhibition of FVa because as has been shown by Nicolaes et al. (J Biol Chem 1995 270:21158-66), FVa cleaved at Arg506 is still partially active as cofactor to FXa, about 40% of its activity being maintained. On the other hand, the slower cleavage at Arg306 results in a complete loss of FVa activity. This Arg 306 cleavage is progressing slowly as is reflected in the slow decrease in FVa activity observed between 5 minutes and 25 minutes of incubation. As is evident from FIG. 4, the variants QGN and SEDY are only slightly better than wt APC, whereas the present variant QGNSEDY is considerably more potent. The present variant QGNSEDY not only yields a very fast drop in FV activity down to approximately 20% FVa activity during the first five minutes but ultimately also inhibits FVa almost completely. These results suggest that the present variant QGNSEDY not only cleaves FVa at Arg506 faster than what is seen for wt APC, but as opposed to wt APC, also cleaves FVa at the Arg306 site.

Experiments similar to those described above (results not shown) were performed, wherein the ability of the variant QGNSEDY and of wt APC to inactivate FVa was compared to this ability of the previously characterized variant GNED (cf. FIG. 1 and FIG. 3). The GNED variant was found to give a curve positioned almost exactly between the curves obtained for other two variants, i.e. GNED was more potent than wt APC bat less efficient than the present variant QGNSEDY. These experiments were all performed without addition of exogenous protein S. The results obtained were consistent with the results of the experiments performed in Example 2(a) and (c) and illustrated in FIG. 1 and FIG. 3, respectively, that also show that the previously disclosed GNED variant has intermediate activity.

### EXAMPLE 4

### Inactivation of FVa by APC

In this example, the concentration of APC was varied and the remaining FVa activity was measured after 10 minutes of incubation using the prothrombinase assay described in Example 3(i).

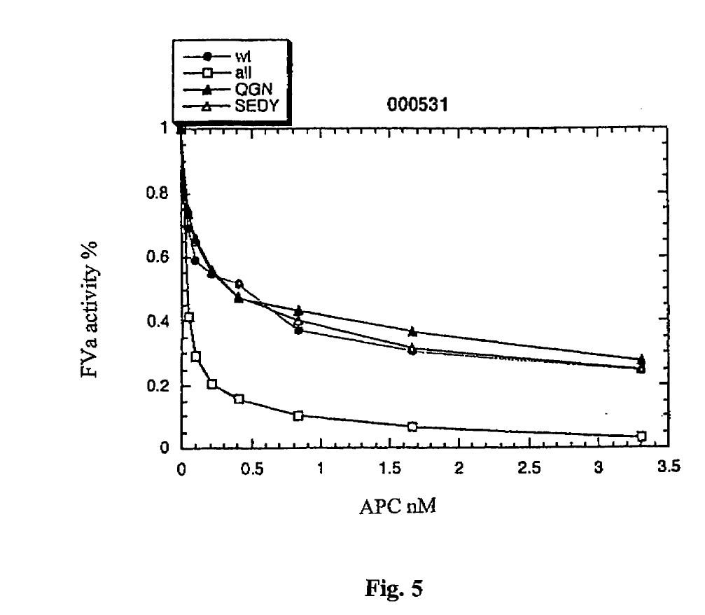

(i) Method: FVa obtained from diluted normal mixed plasma (0.76 nM) was incubated with increasing concentrations of APC (final concentrations given in FIG. 5) and 25 μM phospholipid vesicles (phosphatidylserine/phosphatidylcholine, 10/90, mol/mol) in 25 mM Hepes (pH 7.5), 150 mM NaCl, 5 mM CaCl₂ and 5 mg/ml BSA at 37° C. FVa activity was measured with the prothrombinase assay as described in Example 3(i).

(ii) Results: From FIG. 5, it is evident that these experiments clearly demonstrate the superior efficiency of the mutant ALL, i.e. the present variant QGNSEDY. Even quite low concentrations of APC resulted in a potent inhibition of FVa activity. Moreover, it is obvious from the curves in FIG. 5, tat the mutant ALL not only cleaves at the Arg506 site, which results in an intermediate degradation product of FVa that exhibits about 40% activity but also cleaves at the Arg306 site, which results in an almost complete loss of FVa activity.

### EXAMPLE 5

### Inactivation of Normal and Q506 Mutant FVa by APC

In this example, the normal plasma FVa was replaced with FVa from APC resistant plasma (obtained from an individual with homozygosity for FV:Q506-FV Leiden). This experiment was performed both in the presence and absence of exogenous protein S.

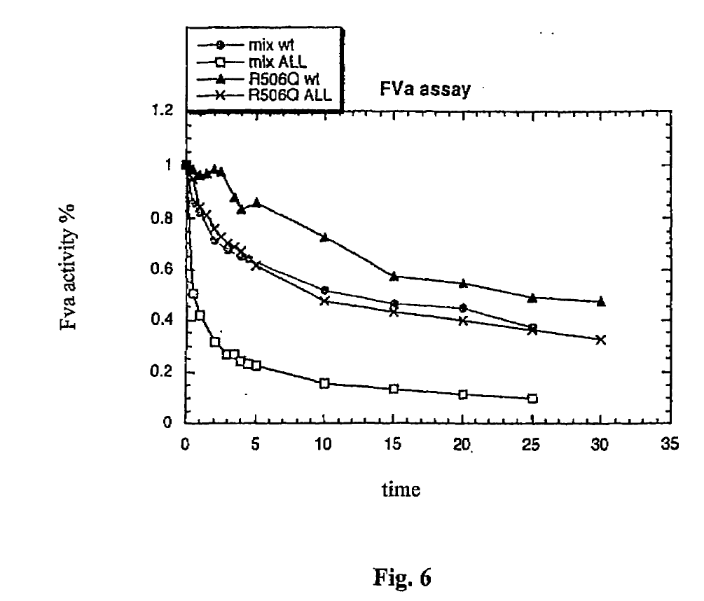

(i) Method: Plasma FVa obtained either from normal pooled plasma or from an individual with homozygous APC resistance (FV:Q506 or FV Leiden) was incubated with 0.4 nM APC and 25 μM phospholipid vesicles as described in Example (3)(i) except that purified. human protein S (100 nM was added to ensue cleavage at Arg 306. At time points as indicated in FIG. 6, remaining FVa activity was determined.

(ii) Results: The addition of wt APC resulted in a slow decrease in FVa activity corresponding to cleavage at Arg306, the slope of the corresponding curve in FIG. 6 being similar to the second part of the curve for wt APC illustrated in FIG. 4. In contrast, the present variant QGNSEDY (or ALL) resulted in a more rapid drop in FV activity consistent with enhanced cleavage of FVa at Arg306 by the AMC variant The addition of protein S enhanced the effect both of wt APC and the QGNSEDY, but yet the difference between the two proteins remained. Thus, it can be concluded that protein S stimulates not only wt APC but also the present APC variant, the latter exhibiting a considerably enhanced binding arty for the phospholipid. This is of interest, since it has been suggested tat protein S functions by enhancing the binding affinity of APC for the phospholipid. If this would be the only mechanism by which protein S works, then one would expect that addition of protein S would decrease the difference between wt APC and the QGNSEDY variant

### EXAMPLE 6

### Membrane Binding Affinity of APC

To investigate the ability of wt and variant protein Cs to bind to phospholipid membranes, the surface plasma resonance technique was used. A commercial variant of this technique is available from BIAcore. In this example, a BIAcore 2000 was used.

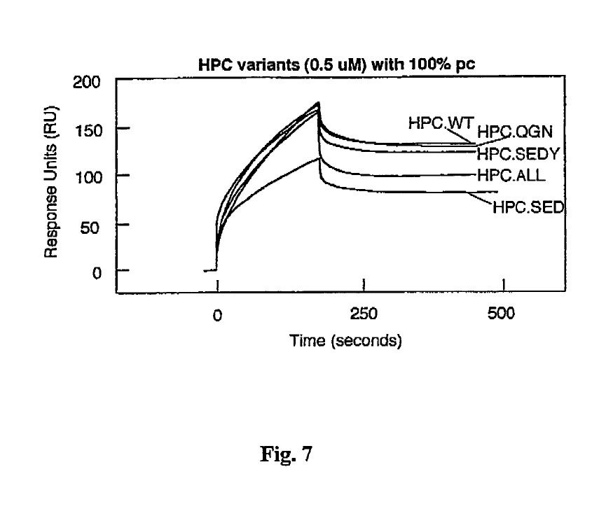

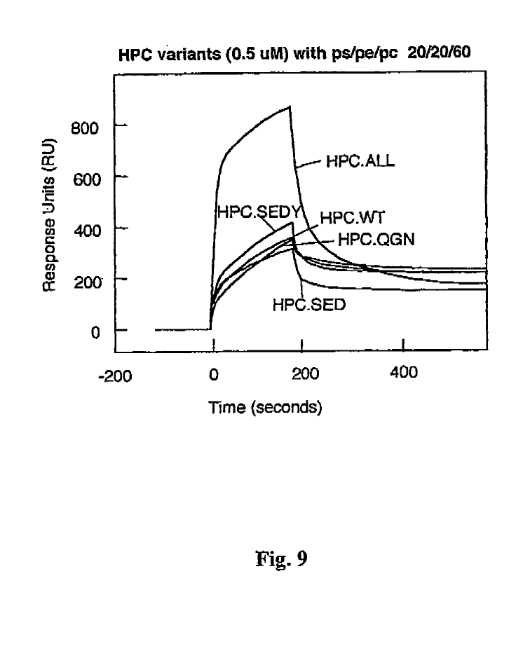

(i) Method: Phospholipid vesicles were captured on the surface of an L1 sensor chip from BIAcore. These chips consist of a dextran hydrogel with covalently coupled hydrophobic aliphatic groups. Three different kinds of vesicles were prepared using extrusion technique (using an Avestin Lipofact basic extrusion apparatus), the three types of vesicles having different phospholipid composition, viz. 1) 100% phosphatidylcholine (FIG. 7), 2) 80% phosphatidylcholine and 20% phosphatidylserine (FIG. (8), and 3) 20% phosphatidylserine, 20% phosphatidylethanolamine and 60% phosphatidylcholine (FIG. 9). Four protein C mutants, viz HPC ALL i. e. QGNSEDY), SEDY, QGN and SED, and wt HPC were tested. In these experiments, the protein C concentration was 0.5 μM and the buffer used, was 10 mM Hepes, 0.15 M NaCl, containing 5 mM CaCl₂, pH 7.5.

Phosphatidylcholine-containing membranes do not bind the vitamin K-dependent proteins unless the negatively charged phosphatidyl serine is part of the membrane. Phosphatidylethanolamine is of particular interest because the presence of this type of phospholipid in the membrane has been shown to enhance the binding of protein C and to enhance the rate of degradation of FVa. Thus, in this example it is investigated whether or not the protein C variants demonstrated changed specificity for the phospholipid types. The different recombinant protein C variants were injected into the BIAcore machine, which had a chip that contained different surface areas covered by the three types of phospholipid membranes.

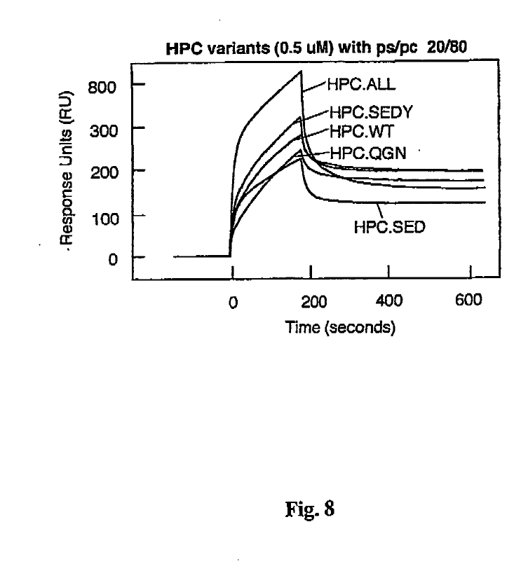

(ii) Results: A concentration of protein C of 0.5 μM was used since, at this concentration, wt protein C is not expected to give any particularly strong binding, because the K_{d} for protein C to negatively charged phospholipid membranes is approximately 15 μM. Thus, in these experiments, it should be possible to see any increased binding ability of the protein C variants. As is evident from FIG. 7, there was very little, if any, binding of the protein C variants to the membrane containing 100% phosphatidylcholine. The maximum response units reached, were only about 160. From FIG. 8, it is obvious that, on membranes containing 20% phosphatidylserine, there was considerably better binding in particular by the present variant QGNSEDY (or ALL) that demonstrated a rapid association of protein C as reflected by the sharp increase in the response as plotted on the Y-axis. The other variants, i.e. QGN, SEDY and SED, behaved like wt protein C. The results shown in FIG. 9, illustrate that the most striking difference between the QGNSEDY (or ALL) variant and the wt protein C was observed when the phosphatidylethanolamine containing membranes were used. The QGNSEDY variant demonstrated a sharp increase in binding to the membrane and very quickly reached a response of about 700 units. During the following 200 seconds, the response rose to approximately 850 response units. The dissociation was followed by discontinuation of the protein C infusion and the bound proteins were relatively quickly released from the membranes. The binding was calcium dependent, since EDTA reversed the binding completely. This behavior is expected from the vitamin K-dependent proteins.

1. A variant blood coagulation component, which is substantially homologous in amino acid sequence to a wild-type blood coagulation component capable of exhibiting anticoagulant activity in the protein C-anticoagulant system of blood and selected from protein C (PC) and activated protein C (APC), said variant component being capable of expressing an anticoagulant activity, which is enhanced as compared to the anticoagulant activity expressed by the corresponding wild-type blood coagulation component, and said variant component differing from the respective wild-type component, in that it contains in comparison with the said wild-type component an amino acid residue modification at position 44 in its N-terminal amino acid residue sequence comprising the first 45 N-terminal amino acid residues and designated the Gla-domain, and that it contains at least one amino acid modification at a position selected from the group consisting of positions 10, 11, 12, 23, 32, and 33 of said Gla-domain.

2. The variant component of claim 1, which has at least 95% amino acid residue sequence identity with the corresponding wild-type component.

3. The variant component of claim 1, which has at least 97% amino acid residue sequence identity with the corresponding wild-type component.

4. The variant component of claim 1, which has at least 98% amino acid residue sequence identity with the corresponding wild-type component.

5. The variant component of claim 1, wherein the amino acid residue modifications are comprised of substituted, deleted or inserted amino acid residues.

6. The variant component of claim 1, wherein said component is a variant PC or a variant APC which exhibits enhanced membrane-binding affinity in comparison with the wild-type component.

7. The variant component of claim 6, which further exhibits enhanced calcium affinity as compared to wild-type protein C.

8. The variant component of claim 1, wherein the said variant component contains at least six, and optionally 7-10, amino acid residue modifications in said Gla-domain.

9. The variant component of claim 1, wherein said variant component contains a modified Gla-domain, which contains the substitution mutations H10Q, S11G, S12N, D23S, Q32E, N33D and H44Y, said modified Gla-domain having the following amino acid sequence:
ANSFLEELRQ GNLERECIEE ICSFEEAKEI FEDVDDTLAF WSKYV (SEQ ID NO:3).

10. (Canceled).

11. The variant component of claim 1, wherein said Gla-domain contains a substitution mutation at a position selected from the group consisting of positions 12, 23, and 44, said substitution mutation being selected from the group consisting of S12N, D23S and H44Y.

12. The variant component of claim 1, wherein said amino acid modifications are located at positions selected from the group consisting of positions 10, 11, 12, 23, 32, 33 and 44 and, optionally, are substitution mutations and wherein optionally all positions 10, 11, 12, 23, 32, 33 and 44 are modified.

13. The variant component of claim 1, wherein said modifications are substitutions.

14. The variant component of claim 9, which variant binds to phospholipid membranes at an association rate that is enhanced in comparison with the wild-type component.

15. The variant component of claim 1, which variant exhibits per se or when activated, an anticoagulant activity determined in vitro in an APTT assay at standard levels of phospholipids, which activity is enhanced in comparison with the wild-type component.

16. The variant component of claim 1, which variant per se or when activated, is capable of degrading a mutated Factor Va that contains the single point mutation Arg 506 Gln.

17. The variant component of claim 9, which variant exhibits per se or when activated, an increased ability to degrade Factor Va in comparison with the wild-type component.

18. The variant component of claim 1, that further contains at least one conservative substitution.

19. The variant component of claim 1, wherein said wild-type blood coagulation component is of human origin.

20. A DNA segment comprising a nucleotide sequence coding for a variant blood coagulation component according to claim 1.

21. A recombinant DNA molecule comprising a replicable vector, which suitably is an expression vector, and a DNA segment according to claim 20 inserted therein.

22. A host cell comprising a microorganism or an animal cell, suitably a cultured animal cell line, harbouring the recombinant DNA molecule of claim 21, which suitably is stably incorporated therein.

23. The host cell of claim 22, which is an adenovirus-transfected human kidney cell.

24. A method for producing a DNA segment of claim 20 coding for a variant blood coagulation component according to claim 1, which comprises:
(a) providing a DNA coding for the wild-type blood coagulation component;
(b) introducing at least one nucleotide modification in said wild-type DNA to form a modified DNA segment coding for a variant blood coagulation component; and
(c) replicating said modified DNA segment.

25. A method for producing a variant blood coagulation component according to claim 1, which comprises:
(a) providing a DNA-segment that codes for the said variant component;
(b) introducing said DNA segment provided in step (a) into an expression vector;
(c) introducing said vector, which contains said DNA segment, into a compatible host cell;
(d) culturing the host cell provided in step (c) under conditions required for expression of said variant component; and
(e) isolating the expressed variant component from the cultured host cell.

26. A pharmaceutical composition comprising an effective amount of a variant blood coagulation component according to any claim 1 and a pharmaceutically acceptable carrier, diluent or excipient.

27. The pharmaceutical composition of claim 26, wherein the variant blood coagulation component is the variant component of claim 16.

28. The composition of claim 26, which contains a further blood coagulation component selected from the group consisting of Protein S and intact Factor V.

29. A diagnostic test system, suitably in kit form, for assaying components participating in the protein C-anticoagulant system of blood, said system comprising a variant blood coagulation component of claim 1.

30. The diagnostic test system of claim 29, wherein the variant blood coagulation component is a variant APC and said test system is a system for assaying functional activity of protein S or intact anticoagulant Factor V.

31. A method for inhibiting coagulation in a patient comprising administering to said patient a physiologically tolerable composition comprising a coagulation-inhibiting amount of a variant blood coagulation component according to claim 1.

32. The method of claim 31, wherein thrombosis is inhibited.

33. The method of claim 32, wherein coagulation is inhibited in an individual having the blood coagulation disorder APC resistance.

34. The method of claim 31, wherein a coagulation-inhibiting amount of the variant component of claim 9 is administered to the patient, which variant binds to phospholipids at an increased association rate and produces an increased anticoagulant activity in comparison with the wild-type component.

35-37. (Canceled)


SEQUENCE LISTING (15 sequences)

SEQ ID NO: 1 - protein, 45 aa, Homo sapiens
ANSFLEELRHSSLERECIEEICDFEEAKEIFQNVDDTLAFWSKHV

SEQ ID NO: 2 - protein, 45 aa, Bovine sp.
ANSFLEELRPGNVERECSEEVCEFEEAREIFQNTEDTMAFWSKYS

SEQ ID NO: 3 - protein, 45 aa, Artificial Sequence
  Description of Artificial Sequence Partial modified human protein C sequence
ANSFLEELRQGNLERECIEEICSFEEAKEIFEDVDDTLAFWSKYV

SEQ ID NO: 4 - DNA, 36 bp, Artificial Sequence
  Description of Artificial Sequence oligonucleotide primer
aaattaatacgactcactatagggagacccaagctt

SEQ ID NO: 5 - DNA, 42 bp, Artificial Sequence
  Description of Artificial Sequence oligonucleotide primer
gcactcccgctccaggttgccttgacggagctcctccaggaa

SEQ ID NO: 6 - DNA, 33 bp, Artificial Sequence
  Description of Artificial Sequence oligonucleotide primer
atagaggagatctgtagcttcgaggaggccaag

SEQ ID NO: 7 - DNA, 33 bp, Artificial Sequence
  Description of Artificial Sequence oligonucleotide primer
cttggcctcctcgaagctacagatctcctctat

SEQ ID NO: 8 - DNA, 36 bp, Artificial Sequence
  Description of Artificial Sequence oligonucleotide primer
gcatttaggtgacactatagaatagggccctctaga

SEQ ID NO: 9 - DNA, 36 bp, Artificial Sequence
  Description of Artificial Sequence oligonucleotide primer
ctggtcaccgtcgacgtacttggaccagaaggccag

SEQ ID NO: 10 - DNA, 36 bp, Artificial Sequence
  Description of Artificial Sequence oligonucleotide primer
cagtgtgtcatccacatcttcgaaaatttccttggc

SEQ ID NO: 11 - protein, 4 aa, Homo sapiens
QGED

SEQ ID NO: 12 - protein, 4 aa, Homo sapiens
GNED

SEQ ID NO: 13 - protein, 4 aa, Homo sapiens
SEDY

SEQ ID NO: 14 - protein, 7 aa, Homo sapiens
QGNSEDY

SEQ ID NO: 15 - protein, 4 aa, Homo sapiens
EGED